(19) United States
(12) Patent Application Publication
(10) Pub. No.: US 2007/0015992 A1
(43) Pub. Date: Jan. 18, 2007

(54) System and method for optoacoustic imaging

(75) Inventors: Robert John Filkins, Niskayuna, NY (US); Peifang Tian, Niskayuna, NY (US); Pavel Alexeyevich Fomitchov, New York, NY (US)

Correspondence Address: GENERAL ELECTRIC COMPANY;GLOBAL RESEARCH, PATENT DOCKET RM. BLDG. K1-4A59, NISKAYUNA, NY, 12309, US

(73) Assignee: General Electric Company

(21) Appl. No.: 11/172,248

(22) Filed: Jun. 30, 2005

(51) Int. Cl. A61B 5/05

(52) U.S. Cl. 600/407

(57) ABSTRACT

A system and method are described for optoacoustic imaging a structural or compositional characteristic of an biological object using a coherent, broad range frequency tunable, electromagnetic radiation source and a pulse shaper to generate a sequence of electromagnetic radiation excitation signals.

### BACKGROUND

The invention relates generally to imaging. The invention particularly relates to optoacoustic imaging.

Optoacoustic imaging techniques typically use electromagnetic signals to generate acoustic waves from an object of interest, which is then measured and processed to retrieve information about the object imaged.

Generally, optoacoustic imaging techniques use single frequency, readily available laser systems to generate ultrasound within an object of interest. But different materials absorb different wavelengths at varied levels.

Biological objects such as tissues are complex and varied in nature. It would be highly desirable to add tissue specificity to optoacoustic imaging techniques, whereby specific parts of a biological system, can be targeted and imaged to enable rapid tomographic imaging with enhanced signal to noise ratio. Similarly, adding material specificity to optoacoustic imaging of composite materials and structures, can enable enhanced level of characterization.

Also, low amplitude and/or broad bandwidth acoustic signals can typically lead to decreased signal to noise ratio (SNR) in acoustic detectors and limit the quality of data acquired through optoacoustic imaging, reducing the ability to detect small features with accuracy and leading to poor resolution in the resulting analysis.

Therefore there is a need for an optoacoustic imaging system with dynamic, and agile control of optical characteristics such as frequency bandwidth, amplitude, shape, timing, and phase of the electromagnetic excitation signal and which can detect features with high accuracy and resolution.

### BRIEF DESCRIPTION

One aspect of the present invention is a system for imaging a structural or compositional characteristic of an object, the system comprising at least one coherent, broad range frequency tunable, electromagnetic radiation source to enable generation of an electromagnetic excitation signal, and at least one pulse shaper to control one or more electromagnetic excitation signal characteristic.

One aspect of the present invention is a system for imaging a structural or compositional characteristic of a biological object, the system comprising at least one coherent, broad range frequency tunable electromagnetic radiation source to enable generation of an electromagnetic excitation signal, at least one pulse shaper to control one or more electromagnetic excitation signal characteristic, an optical probe unit to couple the electromagnetic excitation signal into the biological object, and an acoustic receiver to detect opto-acoustically generated acoustic or waves from the biological object.

Another aspect of the present invention is a method for imaging a structural or compositional characteristic of an object, the method comprising generating a sequence of electromagnetic radiation excitation signals, controlling excitation signal characteristics using a pulse shaper, generating acoustic waves in a biological object by directing the excitation signal at the biological object, and irradiating the biological object, wherein the excitation signal imparting energy to the object, detecting and measuring the generated acoustic waves using at least one acoustic receiver; and determining a structural or compositional characteristic by processing the received acoustic wave signal.

### DRAWINGS

These and other features, aspects, and advantages of the present invention will become better understood when the following detailed description is read with reference to the accompanying drawings in which like characters represent like parts throughout the drawings, wherein:

FIG. 1 is a schematic representation of an optoacoustic imaging system in one embodiment of the present invention.

FIG. 2 is a schematic representation of a coherent electromagnetic radiation source in another embodiment of the present invention.

FIG. 3 is a schematic representation of a pulse shaper in another embodiment of the present invention.

FIG. 4 is a schematic representation of pulse shaper in another embodiment of the present invention.

FIG. 5 is a schematic representation of a pulse shaper in another embodiment of the present invention.

FIG. 6 is a schematic representation of a pulse shaper in another embodiment of the present invention.

FIG. 7 is a schematic representation of a pulse shaper in another embodiment of the present invention.

FIG. 8 is a schematic representation of an optoacoustic imaging system in one embodiment of the present invention.

FIG. 9 is a schematic representation of an optoacoustic imaging system in another embodiment of the present invention.

### DETAILED DESCRIPTION

The term “optoacoustic imaging” or interchangeably “photoacoustic imaging,” as used herein refers to the use of electromagnetic radiation to generate acoustic signal or waves in objects, to image structural or compositional characteristics of the object. In the case of biological objects, the characterization could be done, in vivo or in vitro.

The term “radiation” as described herein refers to electromagnetic radiation of any wavelength or frequency.

The term “imaging” as used herein refers to structural imaging such as tomographic imaging or alternatively to compositional imaging or both.

Optoacoustic imaging techniques typically uses an electromagnetic excitation signal, which is directed at an object. Absorption of radiation by the object results in heat output, leading to a rise in temperature locally, causing thermal expansion. The thermal expansion leads to the generation of pressure waves or acoustic waves, which propagate outward from the source of the heating. The acoustic wave generated is both a function of the material properties of the object, as well as the wavelength of the optical signal used to generate the acoustic wave. A receiver detects the time, magnitude and shape of the received acoustic waves, which are then measured and processed to retrieve information on the structural and compositional features of the object.

As is well known to those skilled in the art, Beer-Lambert law describes the absorption of electromagnetic radiation in a material. The absorption is a function of both material properties as well as wavelength of incident radiation. The behavior of photoacoustic waves generated due to absorption of incident electromagnetic radiation can be modeled using the following equation:

$${∇}^{2} p ( r , t ) - \frac{1}{{v}_{a}^{2}} \frac{{\partial }^{2} p ( r , t )}{\partial {t}^{2}} = - \frac{β}{{C}_{p}} \frac{\partial Q ( r , t )}{\partial t} , \qquad ( 1 )$$

where p(r,t) is acoustic pressure at a time t and position r, vₐ is speed of acoustic waves, β is isobaric volume expansion coefficient, Cₚ is specific heat and Q(r, t) is heat function of the optical energy deposited in the tissues per unit volume per unit time, which can be expressed as
Q(r,t)=A(r)I(t), (2)
where A(r) describes the optical energy deposited in the tissues at a position r and I(t) describes the shape of the irradiation pulse, which can be further expressed as I(t)=δ(t) for impulse heating.

One embodiment of the present invention is an optoacoustic system for imaging structural or compositional features of a biological object. Biological systems, such as human and animal bodies, are made up of different tissue types. Different types of tissues absorb different wavelengths to varied levels. For example, water has significant absorption below about 200 nm and above about 1 micron wavelength range. Some proteins have good absorption below about 300 nm, while a pigment like melanin shows absorption in the 400 to 800 nm range. Hemoglobin and oxygenated hemoglobin have varying absorption levels in the 300 nm to 1 micron range. Also, healthy and diseased tissue of the same type may absorb radiation differently. Further, a tumor may exhibit different absorption characteristics when compared to the tissue substrate it is on. Therefore, it is desirable to have a system that can image different tissue types at their respective peak absorption wavelengths. In one embodiment, the optoacoustic system includes at least one coherent, broad range frequency tunable, electromagnetic radiation source to enable generation of an electromagnetic excitation signal. In one embodiment, the broad range is given by a wavelength range greater than about plus or minus 5 nm about a center wavelength. In a specific embodiment, the broad range is given by a wavelength range greater than about plus or minus 10 nm about a center wavelength. In a more specific embodiment, the broad range is given by a wavelength range greater than about plus or minus 50 nm about the center wavelength.

Another embodiment of the present invention is an optoacoustic system for imaging structural or compositional features of a composite material or structure. A further embodiment of the present system is an optoacoustic imaging system for imaging structural attributes of a manufactured object. The attributes include but are not limited to defects such as delamination, voids, and foreign inclusions, and quality aspects such as numbers of layers, layer thickness, fiber fractions, fiber orientations and porosity.

In some embodiments, the coherent electromagnetic radiation source is a pulsed wave source. Examples of pulsed lasers include but are not limited to Q-switched and mode-locked lasers. In certain embodiments, the electromagnetic radiation source is an ultrafast source, capable of producing picosecond or femtosecond scale pulses. In a still further embodiment, a pulsed source pumping a lasing medium may be used to generate electromagnetic radiation pulses of a desired wavelength. In certain embodiments, the laser pulse widths can be in millisecond, or microsecond, or nanosecond, or picosecond, or femtosecond range. In certain other embodiments, the coherent EM radiation system is a continuous wave system. A non-limiting example of a coherent electromagnetic radiation system is a titanium sapphire crystal laser, which is broad range frequency tunable from about 680 nm to about 1100 nm center wavelength. In another example, a titanium sapphire laser with an intracavity or external cavity frequency doubler can also be used to generate light in the range from about 340 nm to about 550 nm. In a still further example of a broad range frequency tunable, is an optical parametric oscillator (OPO). Parametric oscillators and amplifiers employ nonlinear optical crystals such as but not limited to lithium triborate (LBO), lithium niobate(LiNbO), potassium triphosphate (KTP) and barium borate (BBO). Non-limiting tuning ranges include from about 525 nm to about 665 nm, from about 1050 nm to about 1320 nm, and from about 1350 nm to about 1600 nm. Additionally, the idler wave of such an OPO is typically broadly tunable from about 900 nm to about 2300 nm. Other broad range tunable frequency lasing medium include coloquiriite crystals such as but not limited to Cr:LiSAF (chromium-doped lithium strontium aluminum fluoride), Cr:LiSGAF (chromium-doped lithium strontium gallium aluminum fluoride), Cr:LiCAF (chromium-doped lithium calcium aluminum fluoride), Cr:Forsterite, Cr:YAG, Alexandrite, and Erbium-doped glass.

In one embodiment, the electromagnetic radiation source is configured for a frequency tunable operation range within a wavelength range from about 200 nm to about 2000 nm. In a further embodiment, an electromagnetic radiation emitted by the electromagnetic radiation source is in a wavelength range from about 200 nm to about 2 microns. In a still further embodiment, the electromagnetic radiation wavelength is in a range of about 600 nm to about 1200 nm. In other embodiments, the electromagnetic radiation wavelengths may fall in the radio frequency region, microwave region, X-ray region, or gamma ray region of the electromagnetic spectrum.

The optoacoustic system further includes at least one pulse shaper to control one or more electromagnetic excitation signal characteristics such as but not limited to phase and amplitude. To enable rapid imaging of different types of tissues or materials, it is desirable to generate a sequence of specifically shaped excitation signals. In a further embodiment, the excitation signal wherein the excitation signal comprises a signal sequence. For example, an excitation signal sequence may comprise two short pulses in the picosecond or lower scale range followed by a longer pulse in the microsecond or nanosecond scale range. In one embodiment, excitation signal sequence can be predetermined based on preexisting data regarding the object to be imaged. In another embodiment, the excitation signal sequence is dynamically determined. For example, a probe signal may precede an excitation signal to provide information about the object to be imaged which may be employed in determining the excitation signal sequence. Knowledge of the sequence or order of the excitation signals enables tomographic reconstruction. In one embodiment the pulse shaper is an integral part of the radiation source. In another embodiment the pulse shaper is external to the coherent radiation source.

A pulse can be defined by its intensity and phase in either time or frequency domain.

The pulse in time domain is given by
E(t)=A(t)e^{-jφ(t)}, (3)
where A(t) is the time dependent amplitude and φ is the phase. The pulse in the frequency domain is given by
E(ω)=A(ω)e^{-iφ(ω)}, (4)
where A((ω) and φ(ω) are the amplitude and phase in the frequency domain.

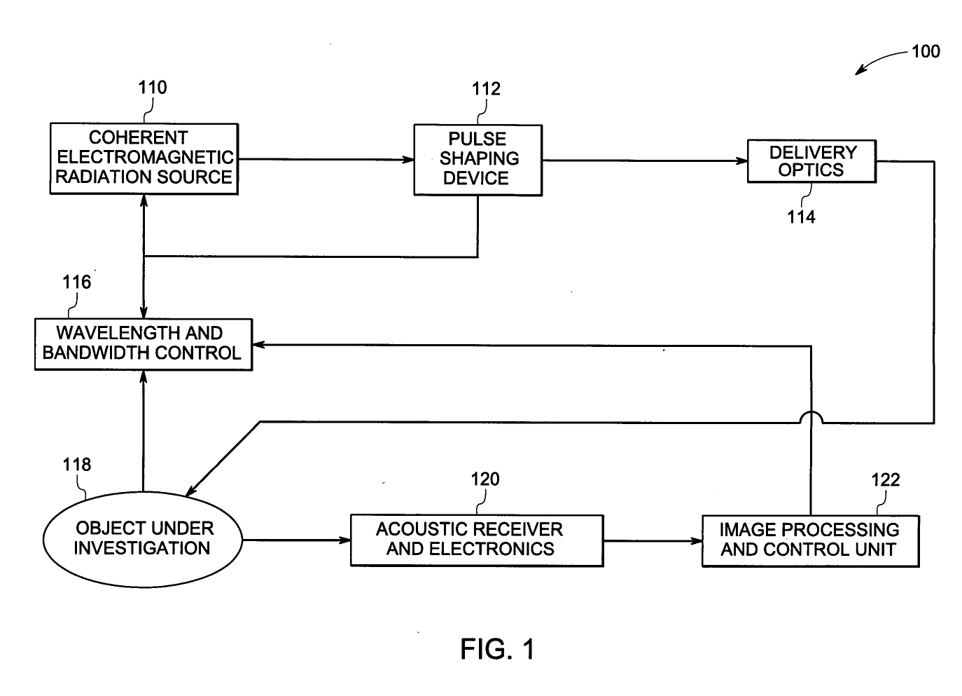

FIG. 1 is a schematic representation of an optoacoustic system in one embodiment of the present invention. The system includes a tunable coherent electromagnetic radiation source 110 such as a laser source. Coherent radiation from the laser is incident on the pulse shaper 112, which modulates the amplitude or phase or both of the incident radiation, and outputs a shaped excitation signal. Delivery optics 114 delivers the excitation signal to the biological object 118 to be imaged and irradiates a region of interest. In a non-limiting example, the delivery optics may include an array of optical probes to deliver the imaging excitation signal to the object. A wavelength and bandwidth control unit 116 may also be present to help dynamically control the frequency and bandwidth of the excitation signal. The wavelength and bandwidth control unit 116 can alternatively be present as an integral part of the laser system 110. Acoustic receiver and electronics 120 detects and measures the generated acoustic signal from the optically activated region of interest, and a processing and control unit 122 processes the measured data for image reconstruction and analysis. Non-limiting examples of acoustic receivers include piezoelectric transducers, electromagnetic transducers, gas-coupled laser acoustic detectors, embedded or surface fiber optic ultrasonic sensors, and optical interferometric detectors.

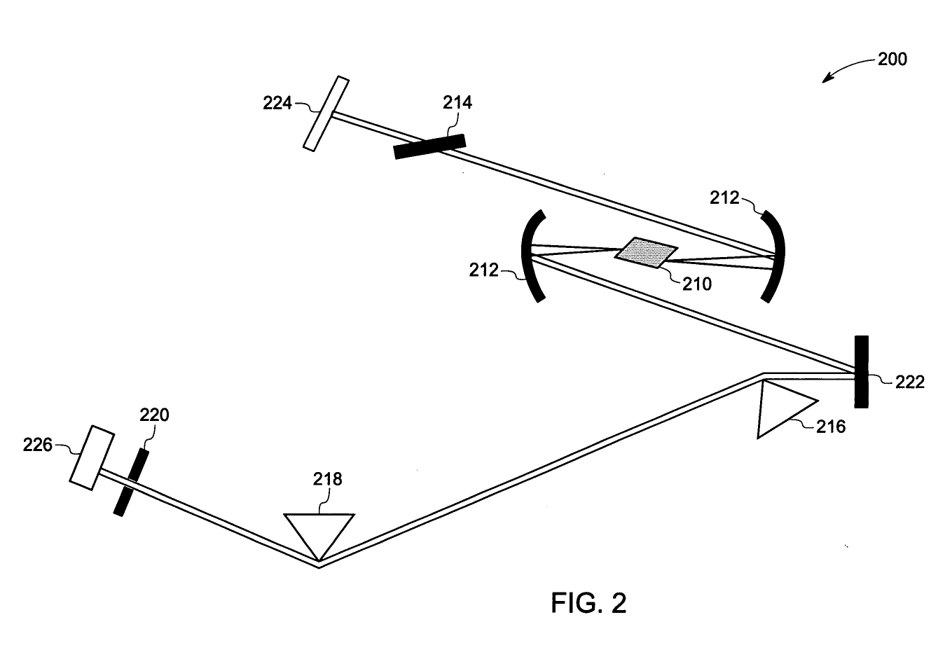

FIG. 2 is a schematic representation of a coherent broad range frequency tunable electromagnetic radiation system 200, in one embodiment of the present invention. A titanium sapphire crystal 210 is placed in an optical cavity defined by cavity mirrors 212. Tuning element 214 is used to tune the frequency output of the laser 200. Examples of tuning elements include but are not limited to filters such as birefringent filters and etalons. The laser cavity typically also includes prisms 216 and 218, motorized slit 220, high reflectors 222, and 224, and output coupler 226. Output coupler 226 enables the coupling out of a fraction of coherent radiation out of the cavity.

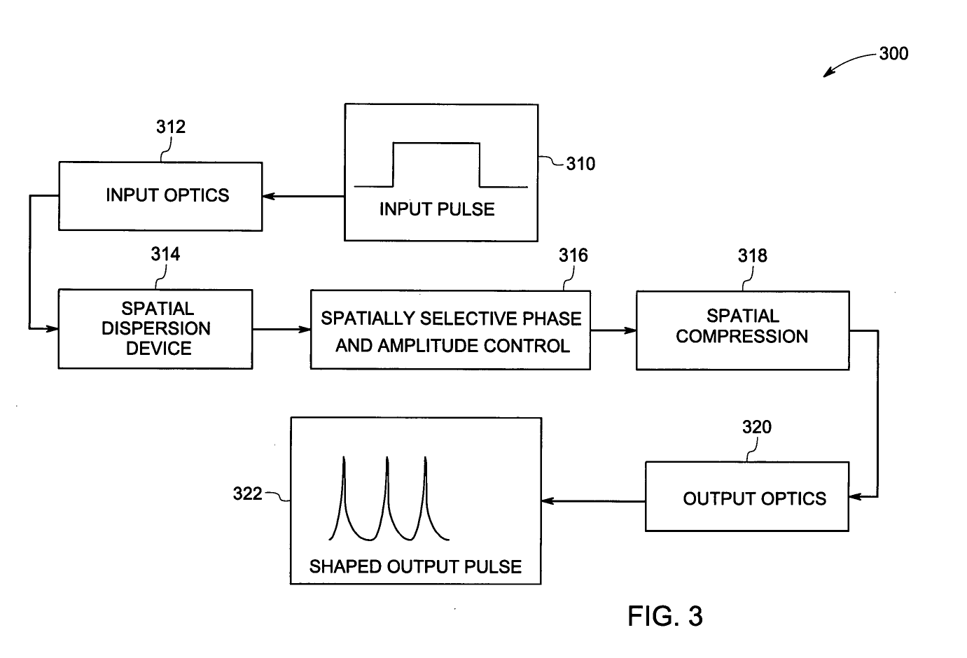

FIG. 3 is schematic representation of a pulse shaper 300 in accordance with another embodiment of the present invention. An input pulse 310 is coupled using input optics 312 into a spatial dispersion device 314, which spatially disperses the different frequency components in the input pulse. The dispersed signal is then incident on a spatially selective phase and amplitude control unit 316, which modulates the phase or amplitude or both of the input signal. The signal is spatially recombined using a spatial compression device 318. The output optics 320 outputs the shaped signal 322.

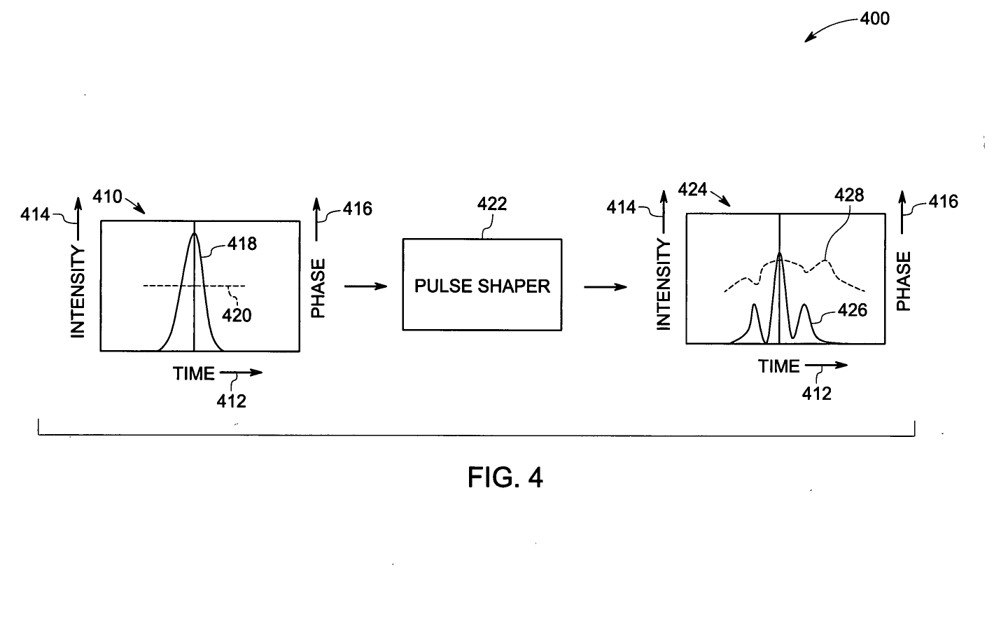

FIG. 4 is a schematic representation of a pulse shaper 400 in another embodiment of the present invention. An input pulse 410 with intensity 414 versus time 412 profile 418, and phase 416 versus time 412 profile 420, is modulated by a pulse shaper 422. When the pulse shaper includes a phase mask, it selectively introduces phase delays for certain wavelengths, and when the pulse shaper includes an amplitude mask it shapes the intensity spectrum in time. A pulse shaped output 424 of the pulse shaper has an intensity profile 426, and a phase profile 428. Non-limiting examples of pulse shapers include spatial masks, spatial light modulators such as acoustooptic modulators, liquid crystal modulators, and deformable mirrors, programmable phase modulators, digital micro mirror devices and grating light valve devices. Pulse shapers can also include one or more optical dispersion elements. Optical dispersion elements include but are not limited to dispersion elements, or compression elements, or any combinations thereof, such as gratings and prisms.

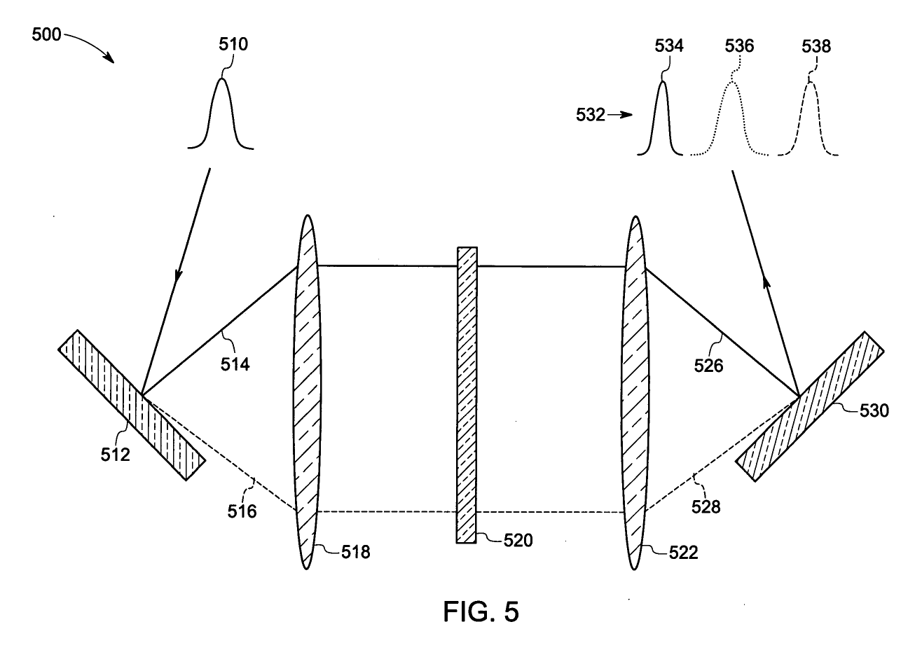

FIG. 5 is a schematic representation of a pulse shaper 500 in a further embodiment of the present invention. An incident broadband optical signal 510 is incident on a grating 512 that disperses the signal, mapping color onto angle. The frequency component with the longest wavelength is dispersed along 514, the component with the shortest wavelength is dispersed along 516, and the dispersed signal is incident on a lens 518. The lens directs the dispersed spectrum onto at least one modulator 520. The modulator 520, in one example, phase delays various frequency components. The modulator 520, in another example can modulate the amplitude. The dispersed and modulated signal is spatially refocused using a lens 522 and spectrally compressed using a grating 530 to give a shaped pulse signal output 532, having frequency components 534, 536, and 538.

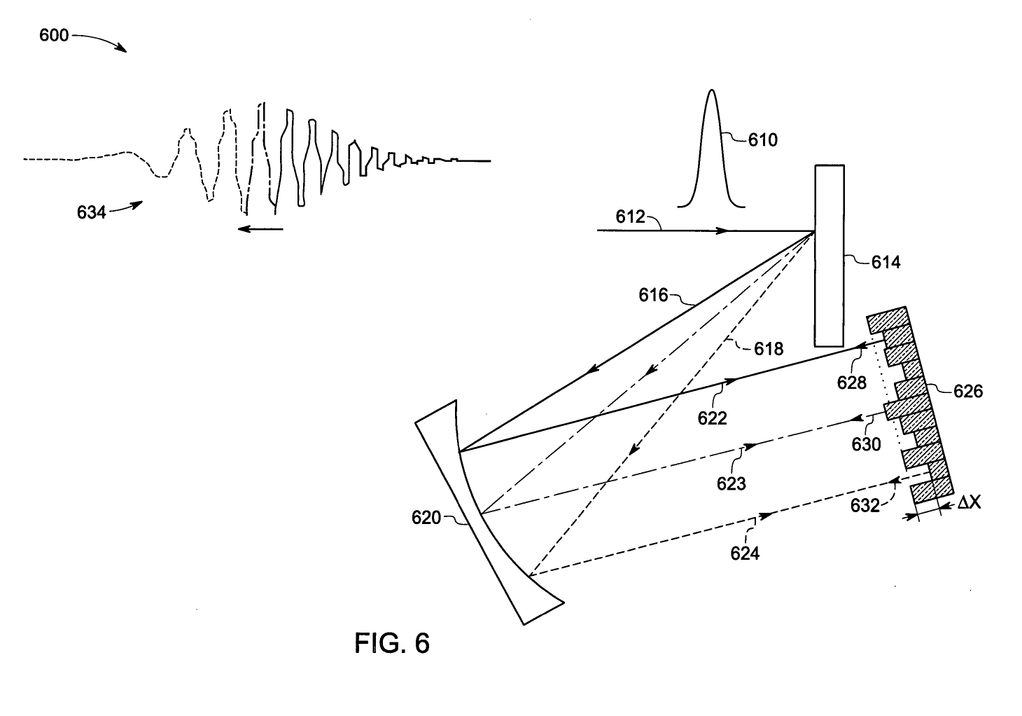

FIG. 6 is a schematic representation of a pulse shaper 600 in another embodiment of the present invention. A signal 610 traverses along 612 and is incident on a grating 614. The signal is spectrally dispersed by the grating 614 along the envelope defined by 616 and 618, and is incident on a spherical mirror 620, which reflects the spectrally dispersed signal along 622, 623, and 624 onto a deformable mirror 626. In some embodiments, the deformable mirror may be pixilated as shown in FIG. 6, while in some other embodiments a continuous deformable mirror may be used. The deformable mirror 626 introduces phase delays corresponding to a path length difference Δx among the frequency components 628, 630 and 632 of the dispersed signal, and reflects back the phase modulated signal 634. The shaped output signal 634 includes frequency components having different phases.

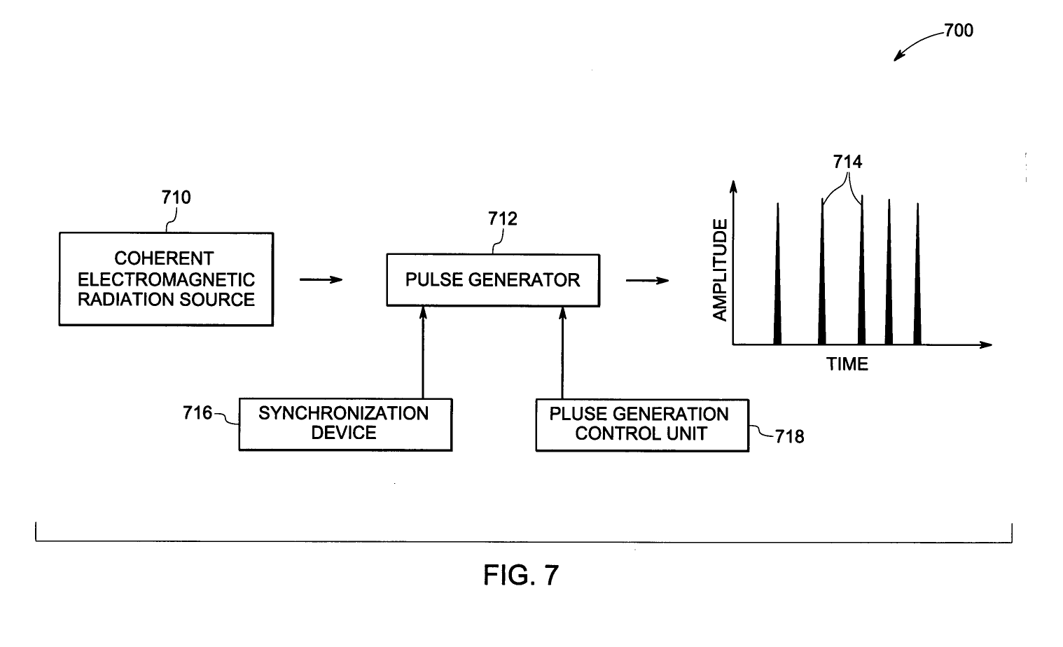

Another embodiment of the present invention is an optoacoustic system including a pulse generator. The coherent electromagnetic radiation source is coupled to a pulse generator to produce a plurality of coherent electromagnetic radiation pulses. FIG. 7 is a schematic representation of a pulse generator 700 in one embodiment of the present invention. Radiation from a coherent source 710 is incident on a pulse generator 712. The pulse generator generates a pulse sequence 714. The pulse generator may be a separate unit or may be part of the coherent electromagnetic radiation source or the pulse shaper. The pulse generator may use one of several techniques to generate pulses including but not limited to q-switching, mode-locking, and chirping.

Although, the pulses depicted in FIG. 7 are triangular spikes, these may be square waves, sinusoidal waves, or any other shape providing the width of the pulses match the above described definition of narrow width or Dirac-like pulses. Additionally, the pulses may be temporally spaced in various arrangements. For example, the pulses may be evenly spaced, unevenly spaced, or distributed in a specific pattern, in time.

Pulse generators include components such as but not limited to Kerr cells, Pockels cells, saturable absorbent media, acoustooptic modulators, shutters, and choppers. In certain embodiments, the system may include additional elements such as but not limited to synchronization devices 716 and pulse generation control unit 718, which typically may include a trigger pulse generator. The synchronization devices are typically used to communicate with a trigger pulse generator to enable adjustment of the Pockels cell to permit photons to pass through a polarizer, thereby generating pulses. The saturable absorbent media generates pulses by saturating with electromagnetic energy until it becomes effectively transparent, permitting electromagnetic energy to pass through. Additional optical elements found in pulse generators include but are not limited to mirrors, output couplers, high reflectors, frequency doublers, and polarizers.

In one embodiment, the pulses generated are Dirac-like pulses. In a non-limiting example, a plurality of pulses with pulse widths of less than or equal to about 20% the time separation between successive pulses is generated. The pulse widths may, by way of example be 10%, 5%, 1% or less of the time separation between successive pulses. Such narrow width pulses may also be termed “Dirac-like” pulses. The advantage of using the Dirac-like pulse of coherent electromagnetic radiation is the higher amplitude of the produced acoustic signal. Dirac-like pulses reduce the bandwidth within various detection frequency ranges while also producing the higher amplitude of the single Dirac-like pulse. The higher amplitude and narrower bandwidth allow better detection of the acoustic signal because the SNR of the acoustic signal is proportional to the amplitude and inversely proportional to the square root of the bandwidth of the acoustic signal.

The higher amplitude and narrower bandwidth of Dirac-like pulses within various frequency ranges leading to low SNR measurements also enables multiple frequency range measurements. From each frequency range, a more accurate measurement can be acquired. Using multiple ranges, information confirming one attribute or simultaneous measurement of multiple attributes may be accomplished.

In certain embodiments of the invention, pulse widths of the coherent electromagnetic radiation excitation signal and the time separation between pulses may be defined and/or controlled. Further, the pulse widths of the coherent electromagnetic radiation excitation signal and the time separation between successive signals may be defined by the physical attribute of the manufactured object or the features of the coherent electromagnetic radiation signal. The pulses may be defined to generate a specific acoustic response in the object.

A further embodiment of the optoacoustic system of the present invention includes a pulse generation control unit 716 to control and modify parameters such as but not limited to pulse widths and the time separation between pulses. The control unit may be internal to the pulse generator or external to it. The control unit may also control these parameters in the coherent radiation source and/or the pulse generator to optimize the acoustic signal generated. The control unit may use structural or compositional attributes of the biological object or the features of the coherent electromagnetic energy pulses to determine proper or optimal pulse widths and time separation between pulses. The control unit may also use this information to determine the time difference between the Dirac-like pulses in a series of Dirac-like pulses of coherent electromagnetic energy. Other characteristics of the Dirac-like pulses such as power, temporal profile, beam shape, beam size, and frequency content may also be controlled. As such, a pulse may be defined to produce a particular acoustic signal or response.

Another embodiment of the present invention is a method for optoacoustic imaging a structural or compositional characteristic of a biological object. The method includes the steps of generating a sequence of electromagnetic radiation excitation signals, dynamically controlling the excitation signal characteristics using a pulse shaper, generating acoustic waves in a biological object by directing the excitation signals and irradiating the biological object, detecting the generated acoustic waves using at least one optoacoustic receiver, and determining a structural or compositional characteristic by processing the acoustic wave signal.

In a more specific embodiment, the method of generating a sequence of electromagnetic excitation signals includes generating a sequence of tissue specific electromagnetic signals. In another embodiment the method includes the step of generating one or more probe signals to determine the optical absorption characteristics of the biological object. The characteristics of the excitation signal can be determined or modified based on the determined optical absorption characteristics. Non-limiting examples of probe signals include but are limited to broadband signals and tone bursts.

In a non-limiting example, a laser may be tuned to an absorption peak of a particular medium for example, deoxygenated hemoglobin. At this operating point, the optoacoustic signal is detected and an image stored. The laser is then rapidly tuned to a second absorption peak, for example oxygenated hemoglobin, by use of a pulse shaper or a bandwidth control device. A second optoacoustic date set is collected. An image is finally synthesized using information from both data sets. More typically, measurements at four or more wavelengths are performed to synthesize an image. It is further possible, using the pulse shaper, to produce a single laser excitation event that comprises two or more pulses of different center wavelength, separated in time by a fixed amount. In a further embodiment the pulses may be designed to generate a specific response from the object to be imaged.

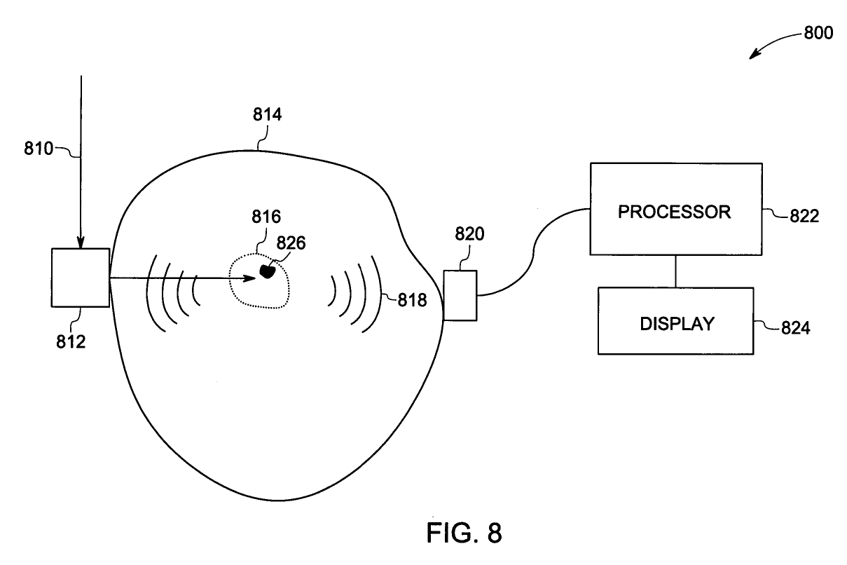

FIG. 8 is a schematic representation of an optoacoustic imaging system 800 in accordance with one embodiment of the present invention. Excitation signal 810 is coupled using a probe 812, into a biological object 814, targeted to irradiate a region 816. A receiver 820 detects acoustic waves 818 originating from the irradiated region. The detected acoustic waves are measured and analyzed by a processor 822 and an image is displayed on the display 824. Typically imaging includes the step of scanning the probe over the biological object to enable imaging from different angles. Detection may be in a forward or backward mode, where the receiver detector is found substantially on the same side as the probe or substantially on the opposite side.

In a still another embodiment, the method includes using at least one contrast agent to image at least part of the biological specimen containing the contrast agent. Contrast agents 826 may be preferentially absorbed by certain parts of the biological object and can be preferentially excited. In another embodiment of the present invention, contrast agents are used to enhance the existing photoacoustic effect in the imaged biological specimen. In a non-limiting example, contrast agents comprise radiation-absorbing components, which are excited on absorption of radiation. Desirably, the excitation energy is converted to thermal energy upon deexcitation of the excited components. In a non-limiting example, the contrast agent may absorb wavelengths in a range from about 200 nm to 2 microns. In some embodiments, the contrast agents are non-specific and typically freely diffuse into the various parts of a system injected into. In other embodiments, the contrast agents are functionalized for preferential absorbance at specific sites. Examples of contrast agents include but are not limited to indocyanine green dyes, cyanine dyes such as but not limited to Cy-3, Cy-5, Cy-7, TexasRed™ (available from Molecular Probes, Inc.), and fluorescent proteins such as but not limited to green fluorescent proteins, cyan fluorescent proteins and yellow fluorescent proteins. Further examples of contrast agents include molecular probes that are tagged with absorption dyes or metal nano-particles. The molecular probes may be specifically targeted at certain tumor types. In one example, a poly-lysine molecular probe is used to target a leaky vasculature.

Contrast agents may further enable light absorption and acoustic wave generation in biological objects that are not normally photoacoustic active. Contrast agents may improve the signal to noise ratio by increasing the amplitude of the acoustic wave generated and enhance better imaging of the biological specimen deeply placed within the body containing the biological specimen. In a still further embodiment, contrast agents may be used to create or enhance selective absorption of radiation in biological specimens such as healthy or diseased organs and facilitate acoustic wave generation. For example, this may enable the detection of malignant tumors. In a still further embodiment contrast agents may also be used to scatter and diffuse optical signals to more uniformly illuminate the target biological object and surrounding tissues or biological material.

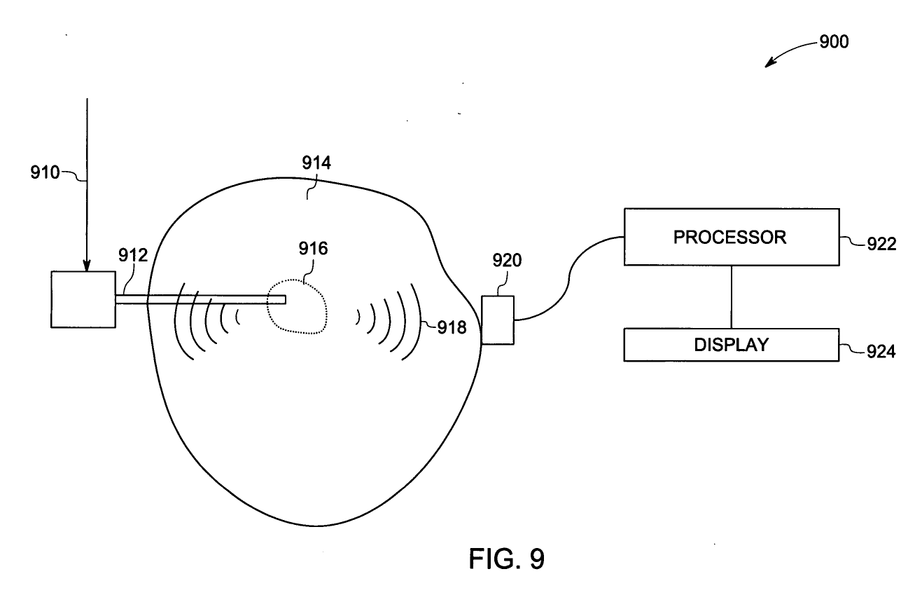

In another embodiment, the step of generating acoustic waves may include the use of an endoscopic probe, wherein the endoscopic probe comprises at least one waveguide such as an optical fiber. FIG. 9 is a schematic representation of an optoacoustic endoscopic imaging system 900 in another embodiment of the present invention. Excitation signal 910 is used to irradiate a target region 916 in a biological object 914 using an optical endoscopic probe 912. A receiver 920 detects the generated acoustic waves 918. The detected acoustic waves are measured and analyzed by a processor 922 and an image is displayed on the display 924.

Although acoustic receivers have been described for purposes of example, other receivers may be used. In some embodiments of the present invention the optoacoustic system and method is used to measure the heat and acoustic energy generated, which is characteristic of the optical properties of the irradiated object such as radiation absorption efficiency and frequency of radiation absorption. In other embodiments the optoacoustic signals are also a measure of one or more physical properties such as elasticity, density, thickness, thermal conductivity and specific heat of the material in which they are generated. In a still another embodiment a focused irradiation spot is used to beneficially provide localized information.

When a continuous wave radiation signal is used, the photoacoustic effects may be analyzed in the frequency domain by measuring amplitude and phase of one or several Fourier components. Alternatively, short pulses (impulses) of radiation may also be employed. When pulses are used, analysis may be made in the time domain, i.e. on the basis of the time taken for the acoustic wave to reach the detector, thus enabling depth profiling. In this case, the absorption of each light pulse and subsequent heating of the various regions of the sample produces one or more positive or negative pressure or acoustic waves that propagate radially from the site of absorption after each pulse. For very short light pulses, the shape of the pressure pulses generated by the light pulses can be determined by the optical and thermal properties, sizes and shapes of the different regions of the sample, the speed of sound within the sites and the surrounding medium, or combinations of such approaches.

In a still further embodiment, the measured acoustic wave is also a measure of the depth of the absorbing targets. Signals from deep within a sample take longer to reach the detector than those from regions near the surface. For pulsed irradiation, the longer transit time translates into a larger separation between the time of arrival of the pulse and the arrival of the signal at the detector. For amplitude-modulated irradiation, the longer transit time translates into a phase change in the detected sound wave.

The elapsed time between the initial irradiation and the arrival of the acoustic waves at the detector provides an indication of the distance of the absorbing site from the receiver. The shape of the detected acoustic wave provides information about the shape of the incident pulse and the shape of the absorbing site. The time-domain signal is equivalent to a distribution of acoustic waves of different frequencies in the frequency domain. The shape of the distribution and the phases of the individual frequencies in the distribution are determined by the length of the irradiating pulse, the shape of the absorbing site, its distance from the point of detection, and the acoustic properties of the medium.

In another embodiment of the present invention, the step of generating includes generating an excitation signal with an intensity varying with a characteristic frequency. This results in a corresponding rise and fall in the pressure imposed on the surrounding medium by the absorbing site. The pressure changes radiate throughout the sample as acoustic waves with fundamental and harmonic frequencies equal to those of the characteristic frequency.

While only certain features of the invention have been illustrated and described herein, many modifications and changes will occur to those skilled in the art. It is, therefore, to be understood that the appended claims are intended to cover all such modifications and changes as fall within the true spirit of the invention.

1. A system for imaging a structural or compositional characteristic of an object, the system comprising:
at least one coherent, broad range frequency tunable electromagnetic radiation source to enable generation of an electromagnetic excitation signal; and
at least one pulse shaper to control one or more characteristics of the electromagnetic excitation signal.

2. The system of claim 1, wherein the electromagnetic radiation source is a continuous coherent electromagnetic radiation source or a pulsed coherent electromagnetic radiation source.

3. The system of claim 1, wherein the electromagnetic radiation source is capable of producing femtosecond or picosecond pulses.

4. The system of claim 1, wherein the electromagnetic radiation source is configured for a frequency tunable operation range within a wavelength range from about 200 nm to about 2000 nm.

5. The system of claim 1, wherein the excitation signal comprises a signal sequence.

6. The system of claim 1, wherein the pulse shaper comprises at least one modulator selected from the group consisting of liquid crystal arrays, spatial masks, spatial light modulators, acoustooptic modulators, deformable mirrors, programmable phase modulators, digital micromirror devices and grating light valve devices.

7. The system of claim 6, wherein the pulse shaper further comprises at least one optical distribution element.

8. The system of claim 6, wherein the optical dispersion element comprises at least one element selected from the group consisting of gratings, prisms, and combinations thereof.

9. The system of claim 1, further comprising a pulse generator configured to produce pulses to enable generation of an electromagnetic pulsed excitation signal.

10. The system of claim 9, wherein the pulse generator comprise at least one component selected from the group consisting of Kerr cells, Pockels cells, saturable absorbent media, acoustooptic generators, and shutters.

11. The system of claim 1, further comprising an acoustic receiver to detect acoustic or pressure waves from the object.

12. The system of claim 11, wherein the receiver comprises a piezoelectric transducer, a gas-coupled laser acoustic detector, an electromagnetic transducers, a fiber optic embedded acoustic sensor, a surface attached acoustic sensor, or any combinations thereof.

13. The system of claim 1, further comprising an optical probe unit configured to couple the electromagnetic excitation signal into the object.

14. The system of claim 13, wherein the optical probe unit comprises an array of optical probes.

15. A system for imaging a structural or compositional characteristic of a biological object, the system comprising:
at least one coherent, broad range frequency tunable electromagnetic radiation source to enable generation of an electromagnetic excitation signal;
at least one pulse shaper to control one or more electromagnetic excitation signal characteristics;
an optical probe unit to couple the electromagnetic excitation signal into the biological object; and
an acoustic receiver to detect opto-acoustically generated acoustic waves from the biological object.

16. The system of claim 15, further comprising a pulse generator configured to produce pulses to enable generation of an electromagnetic pulsed excitation signal.

17. The system of claim 15, wherein the excitation signal comprises a signal sequence.

18. The system of claim 15, wherein the optical probe unit comprises at least one waveguide to guide the optical signal to the biological object.

19. The system of claim 18, wherein the optical probe unit comprises an endoscopic probe.

20. A method for optoacoustic imaging a structural or compositional characteristic of an object, the method comprising:
generating an electromagnetic radiation excitation signal;
controlling characteristics of the excitation signal using a pulse shaper;
generating acoustic waves in an object by directing the dynamically controlled excitation signal at the biological object and irradiating the biological object;
detecting the generated acoustic waves from the object using at least one acoustic receiver; and
determining a structural or compositional characteristic of the object by processing the detected acoustic wave signal.

21. The system of claim 20, wherein the excitation signal comprises a signal sequence.

22. The method of claim 21, wherein the excitation signal sequence is predetermined or dynamically determined.

23. The method of claim 20, further comprising generating one or more probe signals to determine the optical absorption characteristics of the object.

24. The method of claim 23, wherein the one or more probe signals comprise a broadband signal or a tone burst.

25. The method of claim 23, further comprising modifying the excitation signal characteristics based on dynamically determined optical absorption characteristics.

26. The method of claim 20, wherein generating the excitation signal comprises generating a plurality of Dirac-like pulses.

27. The method of claim 26, wherein the Dirac-like pulses have a time separation between the end of a pulse and the beginning of a successive pulse, and have pulse widths of less than or equal to about 20% the time separation.

28. The method of claim 20, wherein detecting comprises detecting the generated acoustic waves in forward mode or backward mode.

29. The method of claim 20, wherein the object is a biological object.

30. The method of claim 29, wherein generating the electromagnetic radiation signal comprises generating a sequence of tissue specific electromagnetic signals.

31. The method of claim 29, wherein imaging comprises tomographic imaging.

32. The method of claim 29, wherein imaging comprises real time monitoring of compositional characteristics.

33. The method of claim 29, wherein imaging comprises in-vivo imaging or in-vitro imaging.

34. The method of claim 29, further comprising using a contrast agent to image at least part of the biological object containing the contrast agent.
